(19) United States
(12) Patent Application Publication
(10) Pub. No.: US 2015/0025335 A1
(43) Pub. Date: Jan. 22, 2015

(54) METHOD AND SYSTEM FOR MONITORING PAIN OF PATIENTS

(71) Applicants: Lakshya JAIN, Los Altos, CA (US); Priya BISARYA, San Diego, CA (US)

(72) Inventors: Lakshya JAIN, Los Altos, CA (US); Priya BISARYA, San Diego, CA (US)

(21) Appl. No.: 14/498,546

(22) Filed: Sep. 26, 2014

Related U.S. Application Data
(63) Continuation-in-part of application No. 14/480,907, filed on Sep. 9, 2014.

(51) Int. Cl. A61B 5/00; A61B 5/11; A61B 5/0402; A61B 5/16; A61B 5/0476; A61B 5/0488; A61B 5/0496; A61B 5/145; A61B 5/0205; A61B 5/0295

(52) U.S. Cl. CPC A61B 5/4824; A61B 5/02055; A61B 5/1102; A61B 5/0402; A61B 5/0295; A61B 5/0476; A61B 5/0488; A61B 5/0496; A61B 5/14542; A61B 5/14532; A61B 5/165; A61B 5/4854; A61B 5/021
USPC 600/301; 600/527; 600/509; 600/526; 600/529

(57) ABSTRACT

The present disclosure provides a method and system for monitoring intensity of pain experienced by one or more users. The method includes measuring the intensity of pain experienced by the one or more users from a pre-determined set of one or more bio-markers using a pre-determined set of one or more bio-sensors, determining a correlation between the one or more bio-markers from the pre-determined set of one or more bio-markers and the intensity of pain experienced by the one or more users, refining the correlation between the one or more bio-markers from the pre-determined set of one or more bio-markers and the intensity of pain experienced by the one or more users by learning from responses of one or more similar users and generating a pain profile for each of the one or more users.

### TECHNICAL FIELD

The present invention relates to the field of biomedical technology and, in particular, relates to monitoring pain of patients to tailor treatments accordingly.

### BACKGROUND

Pain is an unpleasant sensory and emotional experience associated with actual or potential tissue damage. In fact, the pain is a stressor and environment challenge that requires the organism to respond. It is a specific emotion, caused by a stimulus that reflects homeostatic behavioral drive, similar to temperature, itching, hunger, thirst and the like. It may be categorized according to various factors, including type of damage, time for healing and the like. On the basis of healing, the pain can be categorized as a chronic pain and an acute pain. The chronic pain lasts for a longer time as compared to the acute pain. However, both the chronic pain and the acute pain are extremely important problems leading to loss of working capabilities, financial resources and the like.

The pain path classification includes nociceptive pain, neuropathic (pathological) pain and inflammatory pain. The nociceptive pain includes visceral and somatic pain, and the neuropathic (pathological) pain is a disease state caused by damage to the nervous system. It includes peripheral and central neuropathic pain. The inflammatory pain is associated with tissue damage and infiltration of immune cells. The nociceptive pain is a discomfort experienced as a result of an injury. The injury may include but not be limited to a bruise, a cut, a broken bone, appendicitis and the like.

The neuropathic pain is associated with an injury to a nerve or the central nervous system. Such injuries can give rise to paresthesias. For example, the paresthesias may include but not be limited to numbness, tingling, electrical sensations and the like. Further, the neuropathic pain can also generate unusual symptoms. For example, the unusual symptoms may include anesthesia dolorosa in which the area producing the pain is numb to touch. In addition, the nerve, spinal cord and brain injuries can develop syndromes in which ordinarily non-noxious stimuli can cause pain. For example, the syndromes may include but not be limited to allodynia and ordinarily non-noxious stimuli, including but not limited to light pressure or stroking, can cause pain.

Experience of the pain varies from person to person due to inter-individual variability. Moreover, intensity of the pain varies from cause to cause in the individual. Thus, pain management is an extremely important issue. Various factors, directly or indirectly, can contribute in controlling the pain. For example, biological factors (for e.g., sex, genetics and the like), psychological factors (for e.g., mood, attention, distraction and the like), social factors (for e.g., marital status, social support and the like) and the like can significantly modulate intensity as well as unpleasantness caused by the pain.

Presently, it is known that functional interactions exist between systems controlling cardiovascular functions and systems modulating perception of the pain. In addition, there are relationships between pain stimuli and autonomic reactions. In fact, neural structures involved in pain sensation are related to systems performing autonomous control. For example, systolic contraction, systemic resistance, blood pressure (BP), heart rate (HR), heart rate variability (hereinafter ‘HRV’), blood flow, blood pressure, movements due to shifts in central blood mass and myocardial electrophysiological responses and the like. These interactions are important for diagnosing and regulating the pain. For example, the HRV can be used as an important indicator of autonomic nervous system (ANS) reactivity to nociceptive stimulation. Similar arguments can be made in regards to the reaction of other components of nervous system such as, for example, somatic nervous system to the pain stimulus. However, presently, there is no adequate method and system that studies these relationships and the interactions to guide appropriate treatment, care, lifestyle and the like for healthy response in the individuals with respect to the pain.

In light of the above stated discussion, there is a need for a method and system that overcomes the above stated disadvantages. In addition, the method and system should monitor the pain of patients and enable tailoring of treatments accordingly. Further, the method and system should be able to monitor both somatic pain and neuropathic pain whose effects may manifest differently in the body.

### SUMMARY

In an aspect of the present disclosure, a method and system for monitoring intensity of pain experienced by one or more users is provided. The method includes measuring the intensity of pain experienced by the one or more users from a pre-determined set of one or more bio-markers using a pre-determined set of one or more bio-sensors, determining a co-relation between the one or more bio-markers from the pre-determined set of one or more bio-markers and the intensity of pain experienced by the one or more users, refining the co-relation between the one or more bio-markers from the pre-determined set of one or more bio-markers and the intensity of pain experienced by the one or more users by learning from responses of one or more similar users and generating a pain profile for each of the one or more users. The generated pain profile unveils the intensity of pain experienced by the one or more users at various points in body for medical treatment of the one or more users.

The similarity between the one or more users is defined based on a pre-determined set of attributes. The pre-determined set of one or more bio-sensors includes at least one of a ballistocardiogram (BCG) for measuring movements due to shifts in central blood mass of the one or more users, an impedance cardiography (ICG) for detecting blood flow and cardiac output in thorax region of the one or more users using a pre defined set of steps; a dispersion based electrocardiography (ECG) for analyzing myocardial electrophysiological responses of the one or more users using dispersion mapping, a respiration sensor for measuring respiration information of the one or more users and an emotion detector for recording facial emotions of the one or more users experiencing pain.

In an embodiment of the present disclosure, the method utilizes the learned information and the generated profile for monitoring, evaluating and treating the one or more users.

In another embodiment of the present disclosure, the ballistocardiogram (BCG) is analyzed using Google glass (and/or other similar technologies) and measuring of at least one of amplitude, time interval, slopes of BCG waveforms.

In yet another embodiment of the present disclosure, the intensity of pain is measured on a pre-determined scale. The pre-determined scale includes at least one of a visual analog scale (VAS), behavior pain scale (BPS), descriptor differential scale (DDS), dolorimeter pain index (DPI), neck pain and disability scale and physician defined scale.

In yet another embodiment of the present disclosure, the method analyzes the intensity of pain experienced by the one or more users. The intensity is analyzed for each increment between a former pain level and a next pain level.

In yet another embodiment of the present disclosure, the pre-determined set of one or more bio-markers associated with each of the one or more users includes at least one of systolic contraction, systemic resistance, cardiac output including at least one of heart rate, heart rate variability (HRV), blood flow, blood pressure, movements due to shifts in central blood mass and myocardial electrophysiological responses, respiration information, emotions, skin conductance, photoplethysmography (PPG), oxygen saturation, electrocardiography (ECG), electroencephalography (EEG), muscle activity (EMG), accelerometer, EOG, temperature and blood glucose.

In yet another embodiment of the present disclosure, the one or more combinations of one or more bio-sensors from the pre-determined set of bio-sensors are utilized to fetch the pre-determined set of one or more bio-markers associated with each of the one or more users.

In yet another embodiment of the present disclosure, the intensity of pain experienced by the one or more users is increased and measured one or more times on each of the one or more users to check consistency of the learned information with new observations. The new observations are generated when the intensity of pain experienced by the one or more users is increased one or more times.

In yet another embodiment of the present disclosure, a baselining of the one or more users is utilized to treat a new user. The one or more users are most similar to the new user. The similarity is based on at least one of pre-determined set of attributes and pain area.

In yet another embodiment of the present disclosure, the similarity between the one or more users is further defined using at least one of an ayurvedic method of characterization based on VAAT, PITTA, and CUFF or characterization based on Chinese medicines.

In yet another embodiment of the present disclosure, a pre-determined set of grouping scales is utilized for determining personality type and personality group of each of the one or more users. The pre-determined set of grouping scales comprises at least one of tridimensional personality questionnaire test, Dallas pain questionnaire, Roland Morris back pain questionnaire and Wong Baker Faces pain rating scale. The personality group is based on determination of personality traits. The personality traits include at least one of harm avoidance (HA), reward dependence (RD) and novelty seeking (NS). Each of the personality group is analyzed separately for more accurate generation of the pain profile for each of the one or more users. Thus, the pain profile that is generated is tailored according to their known personality type.

In yet another embodiment of the present disclosure, the pain experienced by the one or more users is measured using electrocardiography analysis. The electrocardiography analysis is performed using dispersion analysis under high sampling rate.

In another aspect of the present disclosure, a system for monitoring intensity of pain experienced by one or more users is provided. The system includes a pre-determined set of one or more bio-sensors configured to fetch a pre-determined set of one or more bio-markers associated with each of the one or more users and a communication device. The pre-determined set of one or more bio-sensors includes at least one of a ballistocardiogram (BCG), an impedance cardiography (ICG), a dispersion based electrocardiography (ECG), a respiration sensor and an emotion detector. Further, the communication device includes a pain monitoring application to monitor the intensity of pain experienced by the one or more users.

Furthermore, the pain monitoring application includes an input/output module to receive the pre-determined set of one or more bio-markers associated with each of the one or more users, a display module to display the received pre-determined set of one or more bio-markers associated with each of the one or more users, a diagnostic module to stimulate and measure the intensity of pain experienced by the one or more users, a presentation module to generate a pain profile for each of the one or more users and a database to store the fetched pre-determined set of one or more bio-markers associated with each of the one or more users and the generated pain profile for each of the one or more users. The generated pain profile unveils the intensity of pain experienced by the one or more users for medical treatment of the one or more users.

In an embodiment of the present disclosure, the ballistocardiogram (BCG) is utilized for measuring movements due to shifts in central blood mass of the one or more users, the impedance cardiography (ICG) is utilized for detecting blood flow and cardiac output in thorax region of the one or more users, the dispersion based electrocardiography (ECG) is utilized for analyzing myocardial electrophysiological responses of the one or more users, the respiration sensor is utilized for measuring respiration information of the one or more users and the emotion detector is utilized for recording facial emotions of the one or more users experiencing pain.

In another embodiment of the present disclosure, the system includes a pre-determined scale to rank intensity of pain experienced by each of the one or more users. The pre-determined scale includes at least one of a visual analog scale (VAS), behavior pain scale (BPS), descriptor differential scale (DDS), dolorimeter pain index (DPI), neck pain and disability scale and physician defined scale.

In yet another embodiment of the present disclosure, one or more combinations of one or more bio-sensors from the pre-determined set of one or more bio-sensors is utilized to fetch the pre-determined set of one or more bio-markers associated with each of the one or more users. The one or more bio-sensors are most usable and available bio-sensors.

In yet another embodiment of the present disclosure, the diagnostic module determines a co-relation between the pre-determined set of one or more bio-markers and the intensity of pain experienced by the one or more users.

In yet another embodiment of the present disclosure, the diagnostic module refines the co-relation between the pre-determined set of one or more bio-markers and the intensity of pain experienced by the one or more users by learning from responses of one or more similar users. The similarity between the one or more users is defined based on a pre-determined set of attributes and contributes to a more comprehensive patient pain profile.

In yet another aspect of the present disclosure, a computer system is provided. The computer system includes one or more processors and a non-transitory memory containing instructions that, when executed by the one or more processors, causes the one or more processors to perform a set of steps. The set of steps include measuring the intensity of pain experienced by the one or more users from a pre-determined set of one or more bio-markers using a pre-determined set of one or more bio-sensors, determining a co-relation between the one or more bio-markers from the pre-determined set of one or more bio-markers and the intensity of pain experienced by the one or more users, refining the co-relation between the one or more bio-markers from the pre-determined set of one or more bio-markers and the intensity of pain experienced by the one or more users by learning from responses of one or more similar users and generating a pain profile for each of the one or more users. The generated pain profile unveils the intensity of pain experienced by the one or more users at various points in body for medical treatment of the one or more users.

The similarity between the one or more users is defined based on a pre-determined set of attributes. The pre-determined set of one or more bio-sensors include at least one of a ballistocardiogram (BCG) for measuring movements due to shifts in central blood mass of the one or more users, an impedance cardiography (ICG) for detecting blood flow and cardiac output in thorax region of the one or more users using a pre-defined set of steps, a dispersion based electrocardiography (ECG) for analyzing myocardial electrophysiological responses of the one or more users using dispersion mapping, a respiration sensor for measuring respiration information of the one or more users and an emotion detector for recording facial emotions of the one or more users experiencing pain.

### BRIEF DESCRIPTION OF THE FIGURES

Having thus described the invention in general terms, reference will now be made to the accompanying drawings, which are not necessarily drawn to scale, and wherein:

FIG. 1 illustrates a system showing interaction among various components for monitoring pain of one or more users, in accordance with various embodiments of the present disclosure;

FIG. 2 illustrates a block diagram of a communication device, in accordance with various embodiments of the present disclosure;

FIG. 3 is a flowchart illustrating a method for monitoring the pain of the one or more users, in accordance with the various embodiments of the present disclosure; and

FIG. 4 illustrates a block diagram of the communication device showing additional components, in accordance with various embodiments of the present disclosure.

### DETAILED DESCRIPTION

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

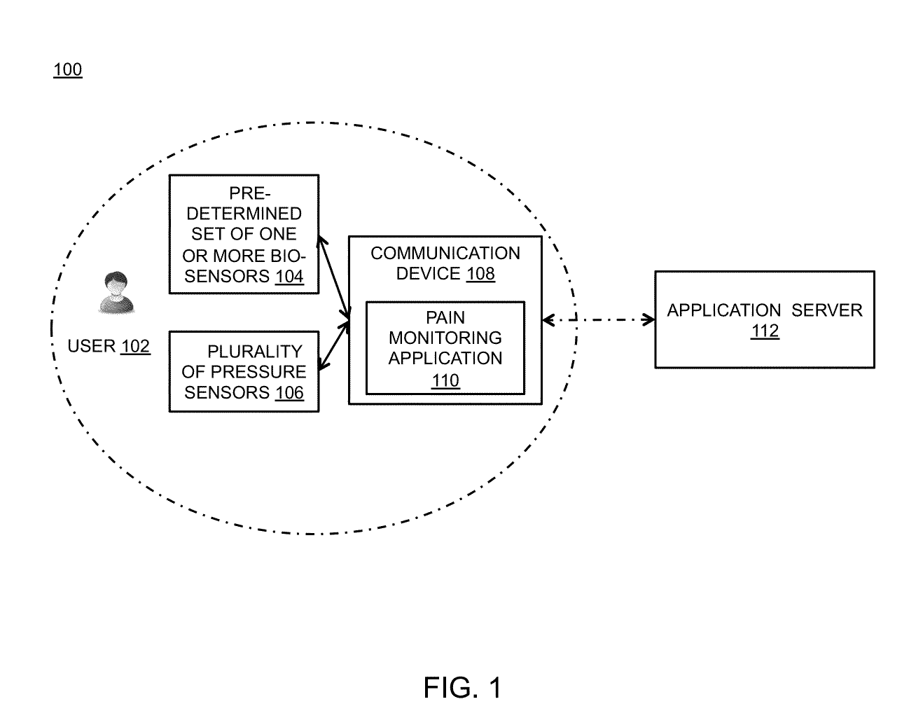

FIG. 1 illustrates a system 100 showing interaction among various components for monitoring pain of users, in accordance with various embodiments of the present disclosure. The system 100 includes a pre-determined set of one or more bio-sensors 104, a plurality of pressure sensors 106 and a communication device 108 associated with a user 102. Examples of the communication device 108 include but may not be limited to mobile phone, laptop, desktop computer, PDA and the like. The communication device 108 executes a pain monitoring application 110. The pain monitoring application 110 monitors the pain of the user 102 and allows tailoring of treatments accordingly. The pain monitoring application 110 communicates with an application server 112 via a network. The user 102 may be a healthy individual or a patient suffering from the pain.

The pre-determined set of one or more bio-sensors 104 fetches a pre-determined set of one or more bio-markers associated with the user 102 including systolic contraction, systemic resistance, cardiac output (for example, heart rate, heart rate variability (HRV), blood flow, blood pressure, movements due to shifts in central blood mass, myocardial electrophysiological responses and the like), respiration information, emotions, skin conductance, photoplethysmography (PPG), oxygen saturation, electrocardiography (ECG), electroencephalography (EEG), muscle activity (EMG), accelerometer, EOG, temperature, blood glucose and the like. The plurality of pressure sensors 106 determines sensitivity of the pain by pressurizing areas of patient's body (body of the user 102) at which the pain is to be diagnosed and recording the pressure level at which the pain is induced in the patient's body.

It may be noted that the plurality of pressure sensors 106 shown in FIG. 1 may be systematically distributed over a specialized pad that can be applied over painful area of the patient's body. Moreover, each pressure sensor from the plurality of pressure sensors 106 is pressed sequentially under control to induce the pain in body of the user 102. Further, location of the specialized pad may be spatially marked with respect to a point on patient's body (for e.g., a particular joint on right/left elbow, right/left knee and the like). This allows for determination of actual point that needs to be checked for treatment or monitoring. Further, it may be noted that the pre-determined set of one or more bio-sensors 104 and the plurality of pressure sensors 106 may be embedded in a wearable component or a device held close to the user 102.

In another embodiment of the present disclosure, the pre-determined set of one or more bio-sensors 104 and the plurality of pressure sensors 106 work in collaboration to enable studying of interaction between pain stimuli and autonomic reactions. The plurality of pressure sensors 106 induces nociceptive stimuli in the body of the user 102 and the pre-determined set of one or more bio-sensors 104 collects the one or more bio-markers from the pre-determined set of one or more bio-markers associated with the user 102 with respect to the pain. Variability in the one or more bio-markers associated with the user 102 corresponds to the autonomic reactions. Examples of the variability in the one or more bio-markers from the pre-determined set of one or more bio-markers include but may not be limited to systolic contraction, systemic resistance, cardiac output (for example, heart rate, heart rate variability (HRV), blood flow, blood pressure, movements due to shifts in central blood mass, myocardial electrophysiological responses and the like), respiration information, emotions, skin conductance, photoplethysmography (PPG), oxygen saturation, electrocardiography (ECG), electroencephalography (EEG), muscle activity (EMG), accelerometer, EOG, temperature, blood glucose, 3-axis accelerometer, 3-axis gyroscope and the like.

In an embodiment, the accelerometers shows greater movement as the user 102 becomes more restless under pain. These movements are picked up sensitively by the 3-axis accelerometer but may not be picked up as accurately by naked human eye. The angular part of movement is picked by the 3-axis gyroscope.

These symptoms (the autonomic reactions) are analyzed by the pain monitoring application 110 to analyze location and intensity of the pain in the body of the user 102. For example, a noticeable and progressively increasing change in certain dimensions of EEG reflects increasing pain which is measured by the pre-determined set of one or more bio-sensors 104.

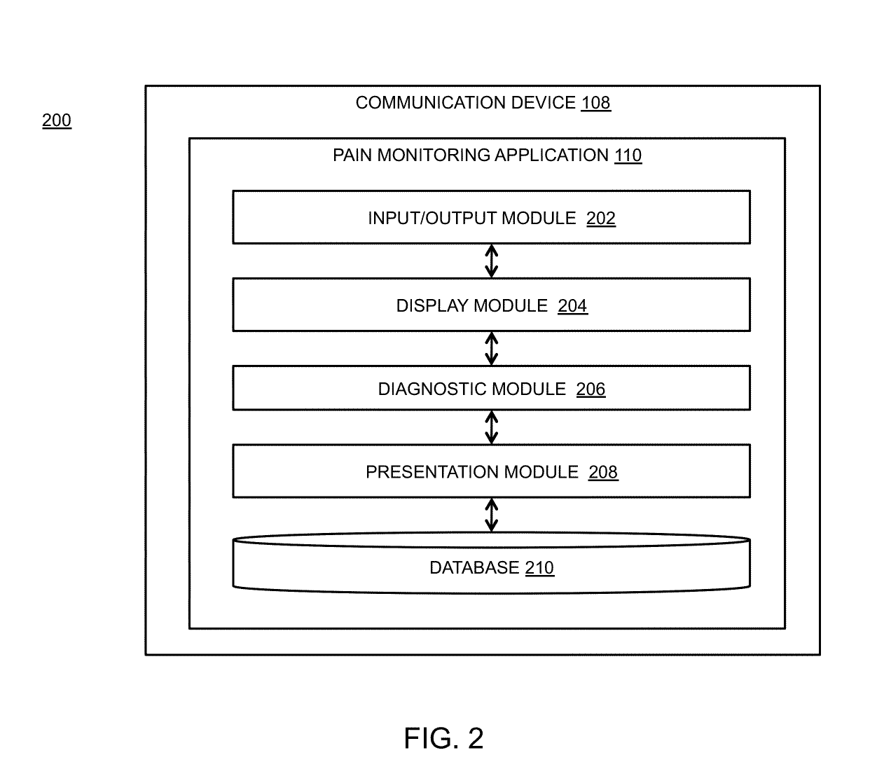

FIG. 2 illustrates a system 200 showing a block diagram of the communication device 108, in accordance with various embodiments of the present disclosure. The communication device 108 executes the pain monitoring application 110. The communication device 108 includes the pain monitoring application 110, an input/output module 202, a display module 204, a diagnostic module 206, a presentation module 208 and a database 210. The input/output module 202 receives the pre-determined set of one or more bio-markers associated with the user 102. The pre-determined set of one or more bio-markers include but may not be limited to the systolic contraction, the systemic resistance, the cardiac output (for example, the heart rate, the heart rate variability (HRV), the blood flow, the blood pressure, the movements due to shifts in central blood mass, the myocardial electrophysiological responses and the like), the respiration information, the emotions, the skin conductance, the photoplethysmography (PPG), the oxygen saturation, the electrocardiography (ECG), the electroencephalography (EEG), the muscle activity (EMG), the accelerometer, the EOG and the like.

The display module 204 displays the received pre-determined set of one or more bio-markers associated with the user 102. The diagnostic module 206 measures the intensity of pain experienced by the user 102 from the pre-determined set of one or more bio-markers using the pre-determined set of one or more bio-sensors 104. The pre-determined set of one or more bio-sensors 104 include a ballistocardiogram (BCG), an impedance cardiography (ICG), a dispersion based electrocardiography (ECG), a respiration sensor and an emotion detector.

The ballistocardiogram (BCG) measures movements due to shifts in central blood mass of said one or more users. The BCG is analyzed by measuring the amplitude, time interval, slope of the BCG waveforms and using Google glass. The Google glass is a type of wearable technology with an optical head-mounted display (OHMD). In an embodiment of the present disclosure, the BCG amplitude decreases with age of the person (user 102). An example of amplitude measure includes I amplitude, I-J amplitude and the like. The I and IJ amplitudes detects subclinical and early abnormalities in large populations, in testing effects of drugs and other therapy, in evaluating certain diseases including aortic valvular disease, in detecting the presence of coronary artery disease, and in predicting life expectancy. Moreover, as the systolic contraction and the systemic resistance are affected by pain, measuring the BCG is a useful marker for measuring the intensity of pain.

The impedance cardiography (ICG) detects blood flow and cardiac output in thorax region of the user 102 using a pre defined set of steps. The pre defined set of steps includes transmission of current through chest of the user 102. The current seeks path of least resistance and the ICG measures the baseline impedance (resistance) to this current. This is because with a change in heartbeat, blood volume and velocity in the aorta, the ICG measures the corresponding change in the impedance. The ICG attributes the large change in impedance to the volumetric expansion of the aorta. Further, the ICG utilizes the baseline and the changes in the impedance to measure and calculate hemodynamic parameters. Moreover, as the cardiac output is likely to change with pain, measuring the impedance cardiography (ICG) is a useful marker for measuring the intensity of pain.

The dispersion based electrocardiography (ECG) analyzes myocardial electrophysiological responses of the one or more users using dispersion mapping. The dispersion mapping is a method used in cardiological clinical practice for detection of the myocardial electrophysiological responses. The ECG Dispersion Mapping is based on analyzing electric micro alternans of the ECG signal. In an embodiment of the present disclosure, a microvolt fluctuation in the ECG morphology is analyzed using ECG dispersion mapping with sophisticated instrumentation and analysis algorithms. The microvolt fluctuation indicates stress due to pain experienced by the user 102.

The respiration sensor measures respiration information of the user 102. The respiration information includes respiratory rate, respiratory rhythm, respiratory effort, inhalation and exhalation metrics, respiratory volume and the like.

The emotion detector records facial emotions of the user 102 experiencing pain. The emotion detector includes but may not be limited to the muscle activity (EMG), 3-axis accelerometer and the like. The EMG detects muscular twitches. The pain experienced by the user 102 produces the muscular twitching. A pre-determined set of methods detects and processes facial expressions. The pre-determined set of methods includes optical flow, hidden Markov model, neural network processing, active appearance model and the like. The EMG provides emotional state by any combination of multimodal recognition (for example, facial expression and speech prosody, facial expressions and hand gestures). In an embodiment of the present disclosure, the facial electromyography recognizes facial gestures. Analyzing facial recognition is useful for monitoring the pain. For example, when a user X is experiencing real pain, the attributes depicting pain include time for which mouth is opened, furrowing between eyebrows, tightening of orbital muscles around eyes, deepening of furrows on both side of nose and the like.

Further, the diagnostic module 206 determines co-relation between the pre-determined set of one or more bio-markers and the intensity of pain experienced by the user 102. Furthermore, the diagnostic module 206 refines the co-relation between the pre-determined set of one or more bio-markers and the intensity of pain experienced by the user 102 by learning from responses of the one or more similar users. The one or more similar users are categorized on the basis of a pre-determined set of attributes. The pre-determined set of attributes includes phenotypical characteristics, genotypical characteristics, mental attributes, physical attributes, mental characteristics, biological characteristics, physiological characteristics, combination of any number of these characteristics and the like.

The diagnostic module 206 analyzes the intensity of pain experienced by the user 102. The intensity is analyzed for each increment between former pain level and next pain level. The presentation module 208 generates a pain profile for the user 102. The generated pain profile unveils the intensity of pain experienced by the user 102 at various points in body for medical treatment of the user 102. The database 210 stores the fetched pre-determined set of one or more bio-markers associated with the user 102 and the generated pain profile for the user 102. The learned information and the generated profile can be utilized for monitoring, evaluating and treating the user 102.

In an embodiment of the present disclosure, the patient (the user 102) may be characterized at different levels including health behavioural, functional, physiological psychological and the like. For each of the patients belonging to these categories, various sub-categories can be created and machine learning can be applied on each of the levels. For example, a set of N users can be partitioned and arranged according to the sub-categories of the levels of characterization.

In another embodiment of the present disclosure, the categories of classification of the one or more users can be combined. For example, if there are 3 categories based on personality type classification system, and 5 categories based on BMI based classification system, then 15 categories can be created based upon combining both the classification systems. Further, each of these 15 categories can be broken into 3 more categories based on ayurvedic system, characterization based on Chinese medicines and the like.

In yet another embodiment of the present disclosure, the pre-determined scale to measure the intensity of pain experienced by the user 102 may include but not be limited to a visual analog scale (VAS), behavior pain scale (BPS), descriptor differential scale (DDS), dolorimeter pain index (DPI), neck pain and disability scale, physician defined scale and the like.

In yet another embodiment of the present disclosure, the pre-determined set of one or more bio-markers associated with the user 102 includes systolic contraction, systemic resistance, cardiac output (for example, heart rate, heart rate variability (HRV), blood flow, blood pressure, movements due to shifts in central blood mass, myocardial electrophysiological responses and the like), respiration information, emotions, skin conductance, photoplethysmography (PPG), oxygen saturation, electrocardiography (ECG), electroencephalography (EEG), muscle activity (EMG), accelerometer, EOG, temperature and blood glucose.

In yet another embodiment of the present disclosure, the intensity of pain experienced by the user 102 is increased and measured several times on the user 102 to check consistency of, and improve upon the learned information with new observations. For example, the new observations are generated when the intensity of pain experienced by the user 102 is increased again one or more times.

In yet another embodiment of the present disclosure, the method is repeated several times to obtain improved machine learning.

In yet another embodiment of the present disclosure, the method envisages a separate training phase for a nerve pain. The training phase is produced through controlled electric shocks.

In yet another embodiment of the present disclosure, a baselining of the one or more users is utilized to treat a new user. The one or more users are the most similar to the new user and the similarity is based on pre-determined set of attributes and pain area. The new user is the person for whom the baselining has not been done.

In yet another embodiment of the present disclosure, a pre-determined set of grouping scales is used for determining personality type and personality group of the user 102. The pre-determined set of grouping scales includes at least one of tridimensional personality questionnaire test, Dallas pain questionnaire, Roland Morris back pain questionnaire, Wong Baker Faces pain rating scale, verbal numerical rating scale (VNRS), verbal descriptor scale (VDS), and brief pain inventory and McGill pain questionnaire.

In yet another embodiment of the present disclosure, the personality group is based on determination of personality traits. The personality traits include at least one of harm avoidance (HA), reward dependence (RD) and novelty seeking (NS).

In yet another embodiment of the present disclosure, the personality group is based on determination of personality traits using any other accepted method of determining such personality trait. This may include 5-factor model, International Personality Item Pool (IPIP) model, or any other model that has been accepted by experts in the field.

In yet another embodiment of the present disclosure, the personality group is analyzed separately for more accurate generation of the pain profile for the user 102.

In yet another embodiment of the present disclosure, stress resulting from the pain experienced by the user 102 is measured using electrocardiography analysis. The electrocardiography analysis is performed using the dispersion analysis under high sampling rate.

In yet another embodiment of the present disclosure, the generated pain profile for the user 102 utilizes pre-defined color coding based on the intensity and location of pain in body of the user 102.

In yet another embodiment of the present disclosure, the generated pain profile for the user 102 is also measured separately for different medical conditions. The different medical conditions may be dental work, fracture, injury and the like.

In yet another embodiment of the present disclosure, intensity of colors used by the presentation module 208 increases proportionally with respect to the pain experienced by the user 102. Moreover, the intensity of colors may differentiate areas in the body of the user 102 that are the most susceptible to the pain. Further, the intensity of colors may segregate affected or bruised areas in the body of the user 102 from the healthy areas.

In another embodiment of the present disclosure, the communication device 108 connects to each of the pre-determined set of one or more bio-sensors 104 and the plurality of pressure sensors 106 via a network (for e.g., LAN, WAN, MAN, Bluetooth, Wi-Fi and the like). Thus, the communication device 108 collects the inputs relating to the autonomic reactions including the heart rate variability, the galvanic skin response, the amount of pressure applied and the like from the user 102.

It may be noted that the pain monitoring application 110 utilizes the pre-determined set of bio-sensors 104 and the plurality of pressure sensors 106 to measure the intensity and the location of the pain. Further, the pain monitoring application 110 maps the pain by generating color coded pain profile/pain model for the user 102. Further, the pain monitoring application 110 may generate tabular report of the pain profile corresponding to results obtained from the diagnostic module 206.

In yet another embodiment of the present disclosure, the pre-determined set of one or more bio-markers associated with the user 102 including the heart rate (HR), the blood pressure (BP), the respiratory rate, the skin conductance and the like may be utilized to track the pain and the location of the pain in the body of the user 102. Moreover, variability in the one or more bio-markers may serve as an indication of the intensity of the pain felt. For example, using an electrocardiography (hereinafter ECG) with extremely high sampling rate enables modeling and analyzing of minute variations in ECG morphology. Similarly, using the respiration rate as the bio-marker, respiratory distress, frequency and depth can be monitored, indicating user's reactivity to the pain. Moreover, an increase in the respiration rate, an increase in shallow breathing or a loss of respiratory rhythm may indicate greater pain. Similarly, reduction in the heart rate variability (HRV) or elevation in the heart rate may indicate severity of the pain. The pain may be modeled and mapped utilizing changes in LF and/or HF spectrum of the heart rate variability (HRV). For example, greater LF (reduced HF) indicates increased perception of pain. Further, the skin conductance can be used to model and map the pain. For example, greater skin conductance measured by the galvanic skin response (GSR) serves as the bio-marker indicating greater pain. Moreover, a noticeable and progressively increasing change in certain dimensions of EEG reflects increasing pain.

In yet another embodiment of the present disclosure, morphology of P-waves may be determined utilizing the pain monitoring application 110 using a number of ECG samples. Size of P-wave determines the extent of an atrial kick representing contraction of atrium where it squeezes extra blood to ventricle (top-off). Since the atrial kick is increased during time of stress, which, in turn, increases during pain, it is a good indicator of the pain. In a normal ECG, the P-wave morphology is captured crudely. However, by high rate sampling (for e.g., a million samples per second) very accurate shape of the P-wave can be captured. Thus, the pain monitoring application 110 may determine extent of the pain felt.

In yet another embodiment of the present disclosure, inducing of the nociceptive stimuli in the user 102 may be achieved by inducing controlled pain by exposing certain areas of the body of the user 102 to heat, cold, electrical stimulation, pressure and the like.

In yet another embodiment of the present disclosure, for one or more or all type of pain stimulus, the stimulus is gradually increased till it reaches the next pain level in the pre-determined scale till the one or more users cannot tolerate it anymore. For each incremental change in the state, all the readings obtained as well as readings obtained in the process of going from the given state to that state are recorded. For example, if a user X reaches in 5 minutes from state 3 to state 4 by application of heat, not only the bio-markers at state 3 and state 4 are recorded but also all the intermediate bio-markers produced in moving from state 3 to state 4 are recorded.

In an example embodiment of the present disclosure, the visual analog scale may present information at discrete intervals about user's bio-metric data. The visual analog scale may be utilized to establish baseline of the user 102 and enables creation of the pain profile for different pain levels using the presentation module 208. Similarly, the pain profile of the user 102 can be created to establish his bio-markers associated with the pain at different values on the visual analog scale. For example, at a value of 3, a first set of values for the HR, the HRV, the BP, the respiratory rate and the like can be annotated. Another set of values can be annotated at a value of 6. Yet another set can be annotated a value of 9, and so on. When the user's bio-markers reach a particular level, then the caretaker or an automated algorithm can extrapolate his pain levels based upon the annotated visual analog scale.

It may be noted that the extrapolation may be effective even though the set of values observed for the bio-markers is not same as the annotated values as long as a bio-marker from the pre-determined set of one or more bio-markers increases or decreases linearly, or even monotonically, along the visual analog scale. It may also be applicable even if there is no monotonic increase or decrease in the one or more bio-markers from the pre-determined set of one or more bio-markers.

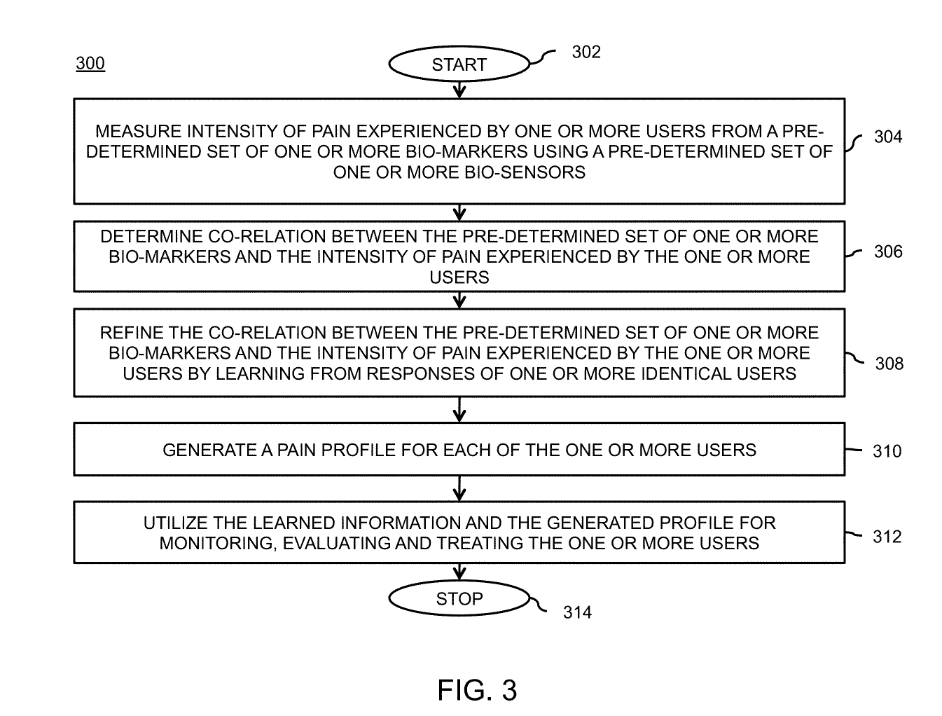

FIG. 3 is a flowchart 300 illustrating a method for monitoring the pain of the users, in accordance with the various embodiments of the present disclosure. The flowchart initiates at a step 302. At a step 304, the diagnostic module 206 measures the intensity of pain experienced by the user 102 from the pre-determined set of one or more bio-markers using the pre-determined set of one or more bio-sensors 104. At a step 306, the diagnostic module 206 determines the co-relation between the pre-determined set of one or more bio-markers and the intensity of pain experienced by the user 102. At a step 308, the diagnostic module 206 refines the co-relation between the pre-determined set of one or more bio-markers and the intensity of pain experienced by the user 102 by learning from responses of the one or more similar users. Following the step 308, at a step 310, the presentation module 208 generates a pain profile for the user 102. Following the step 310, at a step 312, the diagnostic module 206 utilizes the learned information and the generated profile for monitoring, evaluating and treating the user 102. The flowchart terminates at a step 314.

In yet another embodiment of the present disclosure, the pain detection and measurement approach can be improved by a pre-defined process. The pre-defined process includes considering the first set of experiment on a user X where he is subjected to different and increasing levels of pain stimulus and his bio-markers are recorded, and analyzed. (These can be analyzed as related just to the context of given individual, or they can be analyzed in the context of an additional data point in a large group of patients.) However, the analysis may not have repeatability always. For example, next time when the user X is subjected to the same pain stimulus, his bio-markers may change in a different manner. However, this disclosure envisages that the process subjecting a user to pain stimulus can be repeated N times. In each run, the data obtained can be used to enrich model for the given user, and variance in predicted bio-marker values and observed bio-marker values be made smaller and smaller. When the variance becomes smaller than some pre-defined threshold of acceptance then it can be ascertained that the algorithm has reached some type of improved machine learning.

In yet another embodiment of the present disclosure, the user 102 can be assumed to have more than one profile during each run and the user can see the profile that matches him best. One of the profiles can be the person analyzed individually, that is by himself. Another can be when he is analyzed along with everyone in his phenotype, genotype, everyone who has the same personality trait, or everyone who falls into a common group according to ayurvedic classification, or everyone who falls into a given group based upon some pre-defined characteristic. For example, ayurvedic method of characterization based on VAAT, PITTA, and CUFF. Similarly, there may be other methods of characterization based on any other type of systematic classification or distribution of a set of people over multiple groups. Then these profiles can be combined to come up with a more sophisticated profile.

In yet another embodiment of the present disclosure, the pain monitoring application 110 may be used in various fields including dental treatment, medical treatment, sports/athletics, occupational health and the like. For example, a dental hygienist may utilize the pain monitoring application 110 to assist cleaning of patient's teeth.

The dental hygienist may monitor the output provided by the presentation module 208 to identify sudden increase in the bio-markers of the patient and can immediately alter treatment approach and can provide an anesthetic accordingly. Similarly, the pain monitoring application 110 may be utilized for athletic training applications to examine how intensely and frequently an athlete feels the pain in certain areas and for basing treatment according to analysis results provided by the presentation module 208. For example, the pain monitoring application 110 may assist an athlete's recovery from post-game fatigue or bruises by providing a map for where the athlete appears to experience the most pain, upon which the athlete may ice, heat, or otherwise treat the area accordingly. Further, an employer may utilize pain data received from the presentation module 208 of the pain monitoring application 110 accordingly. For example, a construction company may study its workers using the pain monitoring application 110 and may determine discomfort experienced by them in specific areas of their body and may alter the work and tasks of the workers to reduce stress upon that body part. The pain monitoring application 110 allows physicians to understand the location and the intensity of the pain experienced by the user 102 to provide appropriate treatment, care, lifestyle and the like to the user 102.

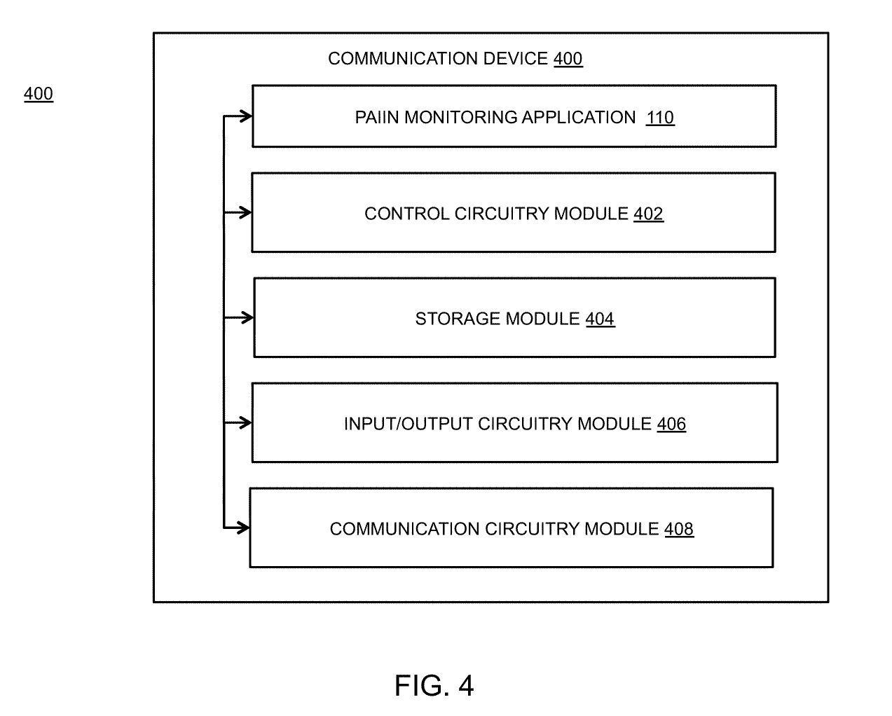

FIG. 4 illustrates a block diagram of a communication device 400, in accordance with various embodiments of the present disclosure. As stated above, in an embodiment, the communication device 400 enables the hosting of the pain monitoring application 110. The communication device 400 includes a control circuitry module 402, a storage module 404, an input/output circuitry module 406, and a communication circuitry module 408. The communication device 400 includes any suitable type of portable electronic device. Examples of the communication device 400 include but may not be limited to a personal e-mail device (e.g., a Blackberry™ made available by Research in Motion of Waterloo, Ontario), a personal data assistant (“PDA”), a cellular telephone, a Smartphone, a handheld gaming device, a digital camera, a laptop computer, and a tablet computer. In another embodiment of the present disclosure, the communication device 400 can be a desktop computer.

From the perspective of this disclosure, the control circuitry module 402 includes any processing circuitry or processor operative to control the operations and performance of the communication device 400. For example, the control circuitry module 402 may be used to run operating system applications, firmware applications, media playback applications, media editing applications, or any other application. In an embodiment, the control circuitry module 402 drives a display and process inputs received from a user interface.

From the perspective of this disclosure, the storage module 404 includes one or more storage mediums including a hard-drive, solid state drive, flash memory, permanent memory such as ROM, any other suitable type of storage component, or any combination thereof. The storage module 404 may store, for example, media data (e.g., music and video files), application data (e.g., for implementing functions on the communication device 400).

From the perspective of this disclosure, the I/O circuitry module 406 may be operative to convert (and encode/decode, if necessary) analog signals and other signals into digital data. In an embodiment, the I/O circuitry module 406 may also convert the digital data into any other type of signal and vice-versa. For example, the I/O circuitry module 406 may receive and convert physical contact inputs (e.g., from a multi-touch screen), physical movements (e.g., from a mouse or sensor), analog audio signals (e.g., from a microphone), or any other input. The digital data may be provided to and received from the control circuitry module 402, the storage module 404, or any other component of the communication device 400.

It may be noted that the I/O circuitry module 406 is illustrated in FIG. 4 as a single component of the communication device 400; however those skilled in the art would appreciate that several instances of the I/O circuitry module 406 may be included in the communication device 400.

The communication device 400 may include any suitable interface or component for allowing the user 102 to provide inputs to the I/O circuitry module 406. The communication device 400 may include any suitable input mechanism. Examples of the input mechanism include but may not be limited to a button, keypad, dial, a click wheel, and a touch screen. In an embodiment, the communication device 400 may include a capacitive sensing mechanism, or a multi-touch capacitive sensing mechanism.

In an embodiment, the communication device 400 may include specialized output circuitry associated with output devices such as, for example, one or more audio outputs. The audio output may include one or more speakers built into the communication device 400, or an audio component that may be remotely coupled to the communication device 400.

The one or more speakers can be mono speakers, stereo speakers, or a combination of both. The audio component can be a headset, headphones or ear buds that may be coupled to the communication device 400 with a wire or wirelessly.

In an embodiment, the I/O circuitry module 406 may include display circuitry for providing a display visible to the user 102. For example, the display circuitry may include a screen (e.g., an LCD screen) that is incorporated in the communication device 400.

The display circuitry may include a movable display or a projecting system for providing a display of content on a surface remote from the communication device 400 (e.g., a video projector). In an embodiment, the display circuitry may include a coder/decoder to convert digital media data into the analog signals. For example, the display circuitry may include video Codecs, audio Codecs, or any other suitable type of Codec.

The display circuitry may include display driver circuitry, circuitry for driving display drivers or both. The display circuitry may be operative to display content. The display content can include media playback information, application screens for applications implemented on the electronic device, information regarding ongoing communications operations, information regarding incoming communications requests, or device operation screens under the direction of the control circuitry module 402. Alternatively, the display circuitry may be operative to provide instructions to a remote display.

In addition, the communication device 400 includes the communication circuitry module 408. The communication circuitry module 408 may include any suitable communication circuitry operative to connect to a communication network and to transmit communications (e.g., voice or data) from the communication device 400 to other devices within the communications network. The communications circuitry 408 may be operative to interface with the communication network using any suitable communication protocol. Examples of the communication protocol include but may not be limited to Wi-Fi, Bluetooth®, radio frequency systems, infrared, LTE, GSM, GSM plus EDGE, CDMA, and quadband.

In an embodiment, the communications circuitry module 408 may be operative to create a communications network using any suitable communications protocol. For example, the communication circuitry module 408 may create a short-range communication network using a short-range communications protocol to connect to other devices. For example, the communication circuitry module 408 may be operative to create a local communication network using the Bluetooth® protocol to couple the communication device 400 with a Bluetooth® headset.

It may be noted that the computing device is shown to have only one communication operation; however, those skilled in the art would appreciate that the communication device 400 may include one more instances of the communication circuitry module 408 for simultaneously performing several communication operations using different communication networks. For example, the communication device 400 may include a first instance of the communication circuitry module 408 for communicating over a cellular network, and a second instance of the communication circuitry module 408 for communicating over Wi-Fi or using Bluetooth®.

In an embodiment, the same instance of the communications circuitry module 408 may be operative to provide for communications over several communication networks. In an embodiment, the communication device 400 may be coupled a host device for data transfers, synching the communication device 400, software or firmware updates, providing performance information to a remote source (e.g., providing riding characteristics to a remote server) or performing any other suitable operation that may require the communication device 400 to be coupled to a host device. Several computing devices may be coupled to a single host device using the host device as a server. Alternatively or additionally, the communication device 400 may be coupled to the several host devices (e.g., for each of the plurality of the host devices to serve as a backup for data stored in the communication device 400).

While the disclosure has been presented with respect to certain specific embodiments, it will be appreciated that many modifications and changes may be made by those skilled in the art without departing from the spirit and scope of the disclosure. It is intended, therefore, by the appended claims to cover all such modifications and changes as fall within the true spirit and scope of the disclosure.

What is claimed is:

1. A method for monitoring intensity of pain experienced by one or more users, said method comprising:
measuring said intensity of pain experienced by said one or more users from a pre-determined set of one or more bio-markers using a pre-determined set of one or more bio-sensors, wherein said pre-determined set of one or more bio-sensors comprises at least one of
a ballistocardiogram (BCG) for measuring movements due to shifts in central blood mass of said one or more users;
an impedance cardiography (ICG) for detecting blood flow and cardiac output in thorax region of said one or more users using a pre-defined set of steps;
a dispersion based electrocardiography (ECG) for analyzing myocardial electrophysiological responses of said one or more users using dispersion mapping;
a respiration sensor for measuring respiration information of said one or more users, wherein said respiration information comprises at least one of respiratory rate, respiratory rhythm, respiratory effort, inhalation and exhalation metrics; and
an emotion detector for recording facial emotions of said one or more users experiencing pain; and a machine learning trained system to recognize the facial grimaces and increasing moistness in eyes
determining a co-relation between said one or more bio-markers from said pre-determined set of one or more bio-markers and said intensity of pain experienced by said one or more users;
refining said co-relation between said one or more bio-markers from said pre-determined set of one or more bio-markers and said intensity of pain experienced by said one or more users by learning from responses of one or more similar users, wherein similarity between said one or more users being defined based on a pre-determined set of attributes; and
generating a pain profile for each of said one or more users, wherein said generated pain profile unveils said intensity of pain experienced by said one or more users at various points in body for medical treatment of said one or more users.

2. The method as recited in claim 1, wherein further comprising utilizing said learned information and said generated profile for monitoring, evaluating and treating said one or more users.

3. The method as recited in claim 1, wherein said ballistocardiogram (BCG) being analyzed using optical head mounted display, and measuring of at least one of amplitude, time interval, slopes of BCG waveforms.

4. The method as recited in claim 1, wherein said intensity of pain being measured on a pre-determined scale, wherein said pre-determined scale comprises at least one of a visual analog scale (VAS), behavior pain scale (BPS), descriptor differential scale (DDS), dolorimeter pain index (DPI), neck pain and disability scale and physician defined scale.

5. The method as recited in claim 1, further comprising analyzing said intensity of pain experienced by said one or more users, wherein said intensity being analyzed for each increment between a former pain level and a next pain level.

6. The method as recited in claim 1, wherein said pre-determined set of one or more bio-markers associated with each of said one or more users comprises at least one of systolic contraction, systemic resistance, cardiac output including at least one of heart rate, heart rate variability (HRV), blood flow, blood pressure, movements due to shifts in central blood mass and myocardial electrophysiological responses, respiration information, emotions, skin conductance, photoplethysmography (PPG), oxygen saturation, electrocardiography (ECG), electroencephalography (EEG), muscle activity (EMG), accelerometer, EOG, temperature and blood glucose.

7. The method as recited in claim 1, wherein one or more combinations of one or more bio-sensors from said pre-determined set of bio-sensors being utilized to fetch said pre-determined set of one or more bio-markers associated with each of said one or more users.

8. The method as recited in claim 1, wherein said intensity of pain experienced by said one or more users being increased and measured one or more times on each of said one or more users to check consistency of said learned information with new observations, wherein said new observations being generated when said intensity of pain experienced by said one or more users being increased said one or more times.

9. The method as recited in claim 1, wherein a baselining of said one or more users being utilized to treat a new user, wherein said one or more users being most similar to said new user, wherein said similarity being based on at least one of pre-determined set of attributes and pain area.

10. The method as recited in claim 1, wherein said similarity between said one or more users being further defined using at least one of an ayurvedic method of characterization based on VAAT, PITTA, and CUFF or characterization based on Chinese medicines.

11. The method as recited in claim 1, wherein a pre-determined set of grouping scales being utilized for determining personality type and personality group of each of said one or more users, wherein said pre-determined set of grouping scales comprises at least one of tridimensional personality questionnaire test, Dallas pain questionnaire, Roland Morris back pain questionnaire, Wong Baker Faces pain rating scale, wherein said personality group being based on determination of personality traits, wherein said personality traits comprises at least one of harm avoidance (HA), reward dependence (RD) and novelty seeking (NS), wherein each of said personality group being analyzed separately for more accurate generation of said pain profile for each of said one or more users.

12. The method as recited in claim 1, wherein said pain experienced by said one or more users being measured using electrocardiography analysis, wherein said electrocardiography analysis being performed using dispersion analysis under high sampling rate.

13. A system for monitoring intensity of pain experienced by one or more users, said system comprising:
a pre-determined set of one or more bio-sensors configured to fetch a pre-determined set of one or more bio-markers associated with each of said one or more users, said pre-determined set of one or more bio-sensors comprises at least one of:
a ballistocardiogram (BCG);
an impedance cardiography (ICG);
a dispersion based electrocardiography (ECG);
a respiration sensor; and
an emotion detector;
a communication device, wherein said communication device further comprises a pain monitoring application to monitor said intensity of pain experienced by said one or more users. The pain monitoring application further comprises:
an input/output module configured to receive said pre-determined set of one or more bio-markers associated with each of said one or more users;
a display module to display said received pre-determined set of one or more bio-markers associated with each of said one or more users;
a diagnostic module configured to stimulate and measure said intensity of pain experienced by said one or more users;
a presentation module configured to generate a pain profile for each of said one or more users, wherein said generated pain profile unveils said intensity of pain experienced by said one or more users for medical treatment of said one or more users; and
a database configured to store said fetched pre-determined set of one or more bio-markers associated with each of said one or more users and said generated pain profile for each of said one or more users.

14. The system as recited in claim 13, wherein said ballistocardiogram (BCG) being utilized for measuring movements due to shifts in central blood mass of said one or more users, said impedance cardiography (ICG) being utilized for detecting blood flow and cardiac output in thorax region of said one or more users, said dispersion based electrocardiography (ECG) being utilized for analyzing myocardial electrophysiological responses of said one or more users, said respiration sensor being utilized for measuring respiration information of said one or more users; and said emotion detector being utilized for recording facial emotions of said one or more users experiencing pain.

15. The system as recited in claim 13, further comprising a pre-determined scale configured to rank intensity of pain experienced by each of said one or more users, wherein said pre-determined scale comprises at least one of a visual analog scale (VAS), behavior pain scale (BPS), descriptor differential scale (DDS), dolorimeter pain index (DPI), neck pain and disability scale and physician defined scale.

16. The system as recited in claim 13, wherein one or more combinations of one or more bio-sensors from said pre-determined set of one or more bio-sensors being utilized to fetch said pre-determined set of one or more bio-markers associated with each of said one or more users, wherein said one or more bio-sensors being most usable and available bio-sensors.

17. The system as recited in claim 13, wherein said diagnostic module being further configured to determine a co-relation between said pre-determined set of one or more bio-markers and said intensity of pain experienced by said one or more users.

18. The system as recited in claim 13, wherein said diagnostic module being further configured to refine said co-relation between said pre-determined set of one or more bio-markers and said intensity of pain experienced by said one or more users by learning from responses of one or more similar users, wherein similarity between said one or more users being defined based on a pre-determined set of attributes.

19. A computer system comprising:
one or more processors; and
a non-transitory memory containing instructions that, when executed by said one or more processors, causes said one or more processors to perform a set of steps, said set of steps comprising:
measuring said intensity of pain experienced by said one or more users from a pre-determined set of one or more bio-markers using a pre-determined set of one or more bio-sensors, wherein said pre-determined set of one or more bio-sensors comprises at least one of
a ballistocardiogram (BCG) for measuring movements due to shifts in central blood mass of said one or more users;
an impedance cardiography (ICG) for detecting blood flow and cardiac output in thorax region of said one or more users using a pre-defined set of steps;
a dispersion based electrocardiography (ECG) for analyzing myocardial electrophysiological responses of said one or more users using dispersion mapping;
a respiration sensor for measuring respiration information of said one or more users; and
an emotion detector for recording facial emotions of said one or more users experiencing pain.
determining a co-relation between said one or more bio-markers from said pre-determined set of one or more bio-markers and said intensity of pain experienced by said one or more users;
refining said co-relation between said one or more bio-markers from said pre-determined set of one or more bio-markers and said intensity of pain experienced by said one or more users by learning from responses of one or more similar users, wherein similarity between said one or more users being defined based on a pre-determined set of attributes; and
generating a pain profile for each of said one or more users, wherein said generated pain profile unveils said intensity of pain experienced by said one or more users at various points in body for medical treatment of said one or more users.
